(19) United States
(12) Patent Application Publication
(10) Pub. No.: US 2023/0210455 A1
(43) Pub. Date: Jul. 6, 2023

(54) IN VIVO BIOMARKERS OF HUMAN LIMBAL STEM CELL FUNCTION

(71) Applicant: The Regents of the University of California, Oakland, CA (US)

(72) Inventor: Sophie Xiaohui Deng, Los Angeles, CA (US)

(73) Assignee: The Regents of the University of California, Oakland, CA (US)

(21) Appl. No.: 18/078,403

(22) Filed: Dec. 9, 2022

Related U.S. Application Data
(60) Provisional application No. 63/288,850, filed on Dec. 13, 2021.

(51) Int. Cl. A61B 5/00; A61B 3/10

(52) U.S. Cl. CPC A61B 5/4842; A61B 3/1005; A61B 5/4005; A61B 5/7246; A61B 5/7264

(57) ABSTRACT

The disclosure provides a comprehensive limbal stem cell deficiency diagnostic and staging system that combines observations of physiological parameters such as clinical presentation, central cornea basal cell density, central corneal epithelial thickness, and total corneal nerve fiber length. It has been discovered that the methodology disclosed herein can both accurately and objectively diagnose limbal stem cell deficiency as well as stage its severity.

### CROSS-REFERENCE TO RELATED APPLICATIONS

This application claims the benefit under 35 U.S.C. Section 119(e) and commonly-assigned U.S. Provisional Patent Application Ser. No. 63/288,850, filed on Dec. 13, 2021, and entitled “IN VIVO BIOMARKERS OF HUMAN LIMBAL STEM CELL FUNCTION” which application is incorporated by reference herein.

### STATEMENT REGARDING FEDERALLY SPONSORED RESEARCH AND DEVELOPMENT

This invention was made with government support under Grant Number EY021797, awarded by the National Institutes of Health. The government has certain rights in the invention.

### TECHNICAL FIELD

Embodiments of the disclosure concern at least the fields of ophthalmology and medicine.

### BACKGROUND OF THE INVENTION

Limbal stem cell deficiency is a clinical syndrome that occurs due to the destruction of limbal stem cells. Limbal stem cells (LSCs) are located in an area of transition between the columnar conjunctival epithelium and the stratified squamous corneal epithelium called “limbus”. Multiple functions are developed in the limbus, such as the nutrition of the peripheral cornea, corneal healing and sensitivity responses. Limbal stem cells which differentiate into corneal epithelium, are essential for maintaining an intact and transparent cornea.(1, 2) Direct damage to LSCs and/or their niche microenvironment leads to limbal stem cell deficiency (LSCD), which is defined as an ocular surface disease in which a decrease in the population and/or function of LSCs leads to an inability to sustain the normal homeostasis of the corneal epithelium.(3, 4)

In the past, the diagnosis of LSCD was predominantly based on the patient's medical history and clinical signs. Impression cytology used to be the “gold standard” method for the diagnosis of LSCD. However, its sensitivity is affected by many factors.(4) More importantly, clinical presentation and impression cytology often are not sufficient to accurately stage the severity of LSCD.(5, 6)

In vivo laser scanning confocal microscopy (IVCM) and anterior segment optical coherence tomography (AS-OCT) have been validated as confirmatory diagnostic tests of LSCD.(4, 7-9) Cellular changes in the cornea and limbus occur in LSCD and can be quantified by both IVCM and AS-OCT.(3, 8, 10, 11) The morphologic changes of epithelial cells in the cornea of eyes with LSCD precede the clinical signs.(8) We have previously shown that basal epithelial cell density (BCD) and epithelial thickness are negatively correlated with the clinical stage of LSCD and can be used to improve the sensitivity in and accuracy of an LSCD diagnosis. (12, 13)

There is a need in the art for additional methods and materials that can better evaluate in vivo parameters useful for diagnosing and staging LSCD.

### SUMMARY OF THE INVENTION

To date, no studies have been performed to systematically evaluate the diagnostic value of certain in vivo parameters and/or to comprehensively analyze which parameters are the most informative for the diagnosis and staging of LSCD. As discussed below, we evaluated a number of different in vivo parameters as biomarkers of limbal stem cell function in order to establish an objective system that detects presence and also the clinical stage of limbal stem cell deficiency in patients with LSCD.

As discussed below, 126 patients (172 eyes) with LSCD and 67 normal subjects (99 eyes) were included in an observational cross-sectional comparative study of physiological parameters having potential correlations with the severity of LSCD. Slit-lamp biomicroscopy, in vivo laser scanning confocal microscopy (IVCM), and anterior segment optical coherence tomography (AS-OCT) were performed to obtain the following: clinical score, cell morphology score, basal cell density (BCD), central corneal epithelial thickness (CET), limbal epithelial thickness (LET), total corneal nerve fiber length (CNFL), corneal nerve fiber density (CNFD), corneal nerve branch density (CNBD), and tortuosity coefficient. Their potential correlations with the severity of LSCD were investigated, and cutoff values were determined. Determination of such scores followed art accepted practices (see, e.g., Aravena et al., Cornea. 2019 January;38(1):1-7.).

Our studies discovered that an increase in LSCD clinical score correlated with a decrease in central cornea BCD, limbal BCD, CET, mean LET, maximum LET, CNFL, CNFD, CNBD, and tortuosity coefficient. Regression analyses showed that central cornea BCD, CET and CNFL were the best parameters to differentiate LSCD from normal eyes (Coef=3.123, 3.379, and 2.223; all p<0.05). The rank correlation analysis showed a similar outcome between the clinical scores and the central cornea BCD (ρ=0.79), CET (ρ=0.82), and CNFL (ρ=0.71). A comprehensive LSCD grading formula based on a combination of these parameters was then established in order to provide a comprehensive staging system combining clinical presentation, central cornea BCD, CET, and CNFL. As discussed below, this comprehensive staging system can accurately and objectively diagnose LSCD and stage its severity.

The invention disclosed herein has a number of embodiments. Embodiments of the invention include, for example, methods of assessing the presence, absence or stage of limbal stem cell deficiency. Illustrative embodiments of the invention include, for example, methods of observing the presence, absence or stage of limbal stem cell deficiency (LSCD) in a patient, Typically, these methods comprise observing in the patient at least one of: central corneal basal cell density; limbal basal cell density; central corneal epithelial layer thickness; mean limbal epithelial layer thickness; maximum limbal epithelial layer thickness; total corneal nerve fiber length; corneal nerve fiber density; corneal nerve branch density; basal epithelial cell morphology; and/or nerve tortuosity coefficient. These methods include correlating this observation with the presence or absence or stage of LSCD in the patient; wherein an increase in a LSCD clinical score correlates with a decrease in central corneal basal cell density, limbal basal cell density, corneal epithelial layer thickness, mean limbal epithelial layer thickness, maximum limbal epithelial layer thickness, total corneal nerve fiber length, corneal nerve fiber density, corneal nerve branch density, and nerve tortuosity coefficient. In this way, the presence or absence, or stage of LSCD in a patient is observed.

Certain embodiments of the invention focus on selected constellations of observations, such as methods that comprise observing a clinical score, a central cornea basal cell density score, a corneal epithelial thickness score and a total corneal nerve fiber length score. In one such embodiment of the invention, the method derives a comprehensive clinical score using a formula: [(clinical score/3)*0.2+central cornea basal cell density score*0.3+corneal epithelial thickness score*0.3+total corneal nerve fiber length score*0.2]*4; wherein: a comprehensive score ≥1 but <5 defines stage 1 LSCD; a comprehensive score ≥5 but <10 defines stage II LSCD; and a comprehensive score ≥10 defines stage III LSCD. In certain embodiments of the invention, method comprises observing at least one of impression cytology images, anterior segment optical coherence tomography images or in vivo laser scanning confocal microscopy images obtained from the patient. In some embodiments of the invention, the method further comprises further comprising administering a therapeutic agent to a patient observed to exhibit the presence of limbal stem cell deficiency (e.g., vitamin A, a methylprednisolone, a loteprednol etabonate, a prednisolone acetate, and/or a cyclosporine or the like).

Embodiments of the invention also include limbal stem cell deficiency (LSCD) diagnostic systems such as those comprising: a processor and a computer-readable program having instructions which cause the processor to assess data obtained from a patient comprising: central corneal basal cell density; limbal basal cell density; central corneal epithelial layer thickness; mean limbal epithelial layer thickness; maximum limbal epithelial layer thickness; total corneal nerve fiber length; corneal nerve fiber density; corneal nerve branch density; basal epithelial cell morphology; and nerve tortuosity coefficient; and then correlate the observation with the presence or absence or stage of LSCD in the patient. In typical embodiments of the invention, the processor uses an algorithm to calculate a LSCD clinical score. In certain embodiments of the invention, the processor uses an algorithm to identify one or more treatment options for a patient having the calculated LSCD score.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

The figures show illustrative aspects and embodiments of the invention.

FIG. 1. Schematic diagram of the severity grading of LSCD. The grading system is composed of 3 parts. The first part is limbus involvement (A). A score of 1 to 4 points is based on the number of clock hours of the affected limbus. One to three hours of limbal range affected is assigned 1 point; 4 to 6 hours, 2 points; 7 to 9 hours, 3 points; and 10 to 12 hours, 4 points. The second part is the cornea surface area affected (B). The 8-mm central cornea surface (within the red ring) is divided into 4 areas according to the long axis of the corneal lesions. Based on the location of involvement, 1 affected area is assigned 1 point; 2 affected areas, 2 points; 3 affected areas, 3 points; and 4 affected areas, 4 points. The third part is the involvement of the visual axis (C). The visual axis area is defined as a central 4-mm circle. If this area is involved, 2 points were assigned; if the area is not involved, then 0 point is assigned.

FIG. 2. Heatmap of the correlation analysis among all parameters in the control and limbal stem cell deficiency groups. The heatmap is a color visualization of correlation analysis. The red and blue dots mean positive and negative correlation, respectively. The stronger the correlation is, the darker the color and the larger size of the dot are. A). The heatmap showed that among all subjects the clinical severity subscore had a strong positive correlation with cell morphology and a negative correlation with basal cell density (BCD, both central corneal and limbus), epithelial thickness (both central corneal and limbus), total corneal nerve fiber length (CNFL), and corneal nerve fiber density (CNFD). B). The analysis of subjects with LSCD only showed that clinical severity had very strong negative correlation with only 4 parameters: BCD at the central cornea, cell morphology at the central cornea, central corneal epithelial thickness (CET) measured by anterior segment optical coherence tomography (AS-OCT), and CNFL.

FIG. 3. The efficacy of all parameters in differentiating between control eyes and eyes with limbal stem cell deficiency. Each red circle represents one parameter. The circles at higher position in the figure indicates a better efficacy to differentiate control eyes from eyes with limbal stem cell deficiency (LSCD). Basal epithelial cell density (BCD), corneal epithelial thickness (CET) and nerve fiber length at central cornea (CNFL), and superior limbal epithelial thickness measured by AS-OCT were the best parameters for differentiating between normal eyes and eyes with LSCD and in staging LSCD.

FIG. 4. Two cases of limbal stem cell deficiency assessed using the comprehensive grading scale. Two cases are presented here to show how the comprehensive grading system is better than clinical grading alone. The slit lamp photos under white light and cobalt blue light, the in vivo confocal microscopic (IVCM) images of corneal basal epithelium and subbasal nerve plexus, and anterior segment optical coherence tomography (AS-OCT) images of central cornea were presented here. Case I (A-E) was a representative case with its clinical severity underestimated by the clinical grading alone. Although no obvious abnormalities were visible in the slit lamp photo taken under white light (A), fluorescein staining revealed late staining from 12 o'clock to 3 o'clock without visual axis involvement, which was outlined with orange arrows (B). The clinical grade would classify the disease at stage I. IVCM identified a reduction in basal epithelial cell density (BCD, C) and nerve fiber length at central cornea (CNFL, E) at the central cornea. AS-OCT (D) showed a transparent hyporeflective epithelium with normal thickness at the unaffected inferior-nasal quadrant (white arrow). In the affected superior-temporal quadrant, the epithelium becomes thinner with increased reflectivity (red arrow). Central corneal epithelial thickness (CET) also reduced in spite of normal reflectivity (yellow arrow). The eye was classified as a stage II disease using the comprehensive grading method. Case II (F-K) was a representative case with its clinical severity overestimated by the clinical grading alone. Although peripheral pannus, stromal opacity, and corneal neovascularization were present (F), fluorescein staining was visible only in the peripheral nasal peripheral cornea and nasal limbal quadrant (G). Goblet cells were also found by IVCM (H) within the area shown by the white frame in F. The eye would be classified as a stage II disease. The reflectivity of corneal epithelial layer is normal, as shown by AS-OCT, and CET was normal despite epithelial irregularity caused by subepithelial scarring (J). IVCM showed normal cell morphology at the central cornea and a slight reduction of BCD (I) and CNFL (K). The eye would be reclassified as a stage I limbal stem cell deficiency.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention.

The corneal epithelium is a stratified squamous epithelium from which superficial terminal cells are naturally shed. Limbal stem cell deficiency (LSCD) is characterized by a loss or deficiency of the stem cells in the limbus that are vital for re-population of the corneal epithelium and to the barrier function of the limbus. When these stem cells are lost, the corneal epithelium is unable to repair and renew itself. This results in epithelial breakdown and persistent epithelial defects, corneal conjunctivalization and neovascularization, corneal scarring, and chronic inflammation. All of these contribute to loss of corneal clarity, potential vision loss, chronic pain, photophobia, and keratoplasty failure.

The loss or dysfunction of limbal stem cells of the corneal epithelium in a sufficient number translates into the incapacity to maintain the dynamic equilibrium of the corneal epithelium and into the onset of LSCD. When this occurs, and to prevent an epithelial defect, there is an invasion of the conjunctival epithelium in the cornea which, in the absence of blood vessels, adopts a phenotype similar to the corneal phenotype although it never manages to transdifferentiate completely; this process is normally accompanied by subepithelial vascularization, with persistent epithelial defects and stromal healing. In more severe cases persistent epithelial defects, calcifications, stromal ulcers and even perforations occur. Clinically, there is a loss of transparency of the cornea which, if it affects its central area, causes a decrease in visual acuity. For discussions of LSCD, see, e.g., Liang et al., Am J Ophthalmol. 2020 August;216:132-139; Le et al., Cornea. 2018 Aug;37(8):1067-107; Chuephanich et al., Cornea. 2017 March;36(3):347-352; Chan et al., Am J Ophthalmol. 2015 October;160(4):678-84; Chan et al., Am J Ophthalmol. 2015 October;160(4):669-77; and Deng et al., Arch Ophthalmol. 2012 April; 130(4):440-5, the contents of which are incorporated by reference.

As discussed in the Examples below, we have developed a diagnostic and staging system for LSCD that combines observations of phenomena such as clinical presentation, central cornea basal cell density, central corneal epithelial thickness, and total corneal nerve fiber length. It has been discovered that this methodology can both accurately and objectively diagnose limbal stem cell deficiency as well as stage its severity.

As illustrated below, the invention disclosed herein has a number of embodiments. Embodiments of the invention include, for example, methods of assessing the presence, absence or stage of limbal stem cell deficiency. Illustrative embodiments of the invention include, for example, methods of observing the presence, absence or stage of limbal stem cell deficiency (LSCD) in a patient, Typically, these methods comprise observing in the patient at least one of: central corneal basal cell density; limbal basal cell density; central corneal epithelial layer thickness; mean limbal epithelial layer thickness; maximum limbal epithelial layer thickness; total corneal nerve fiber length; corneal nerve fiber density; corneal nerve branch density; basal epithelial cell morphology; and/or nerve tortuosity coefficient. These methods include correlating this observation with the presence or absence or stage of LSCD in the patient; wherein an increase in a LSCD clinical score correlates with a decrease in central corneal basal cell density, limbal basal cell density, corneal epithelial layer thickness, mean limbal epithelial layer thickness, maximum limbal epithelial layer thickness, total corneal nerve fiber length, corneal nerve fiber density, corneal nerve branch density, and nerve tortuosity coefficient. Typically, the method comprises correlating the observation with the stage of LSCD (I, II or III) in the patient. In this way, the presence or absence or specific stage of LSCD in a patient is observed.

Certain embodiments of the invention focus on selected constellations of observations. For example, in some embodiments of the invention, the method comprises correlating observations of basal cell density, corneal epithelial thickness and total corneal nerve fiber length with the presence or absence, or stage of LSCD in the patient. In some embodiments, the methods comprise observing a clinical score, a central cornea basal cell density score, a corneal epithelial thickness score and a total corneal nerve fiber length score. Optionally the method comprises observing at least 2 or 3 of: limbal basal cell density; epithelial layer thickness; basal epithelial cell morphology; or corneal nerve branch density. In one embodiment of the invention, the method derives a comprehensive clinical score using a formula: [(clinical score/3)*0.2+central cornea basal cell density score*0.3+corneal epithelial thickness score*0.3+total corneal nerve fiber length score*0.2]*4; wherein: a comprehensive score ≥1 but <5 defines stage 1 LSCD; a comprehensive score ≥5 but <10 defines stage II LSCD; and a comprehensive score ≥10 defines stage III LSCD. In certain embodiments of the invention, method comprises observing at least one of impression cytology images, anterior segment optical coherence tomography images or in vivo laser scanning confocal microscopy images obtained from the patient. In some embodiments of the invention, the method further comprises further comprising administering a therapeutic agent to a patient observed to exhibit the presence of limbal stem cell deficiency (e.g., vitamin A, a methylprednisolone, a loteprednol etabonate, a prednisolone acetate, and/or a cyclosporine or the like).

Embodiments of the invention include a limbal stem cell deficiency (LSCD) diagnostic system comprising: a processor and a computer-readable program having instructions which cause the processor to assess data obtained from a patient comprising: central corneal basal cell density; limbal basal cell density; central corneal epithelial layer thickness; mean limbal epithelial layer thickness; maximum limbal epithelial layer thickness; total corneal nerve fiber length; corneal nerve fiber density; corneal nerve branch density; basal epithelial cell morphology; and nerve tortuosity coefficient; and then correlate the observation with the presence or absence or stage of LSCD in the patient. In typical embodiments of the invention, the processor uses an algorithm to calculate a comprehensive LSCD clinical score. In certain embodiments of the invention, the processor uses an algorithm to identify one or more treatment options for a patient having the calculated LSCD score (e.g., administration of a composition comprising vitamin A, a methylprednisolone, a loteprednol etabonate, a prednisolone acetate, and/or a cyclosporine). Typically, an algorithm derives a comprehensive clinical score using a formula as follows: [(clinical score/3)*0.2+central cornea basal cell density score*0.3+corneal epithelial thickness score*0.3+total corneal nerve fiber length score*0.2]*4; wherein: a comprehensive score ≥1 but <5 defines stage 1 LSCD; a comprehensive score ≥5 but <10 defines stage II LSCD; and a comprehensive score ≥10 defines stage III LSCD.

Further aspects and embodiments of the invention are discussed in the following examples.

### Example 1: Biomarkers of In Vivo Limbal Stem Cell Function

### Methods

This observational cross-sectional comparative single-center study was conducted in accordance with the Declaration of Helsinki and was approved by the Institutional Review Board at the University of California, Los Angeles. Written informed consent was obtained from all subjects per protocol. A total of 126 subjects diagnosed with LSCD based on clinical presentations, the existence of conjunctival epithelium on the cornea under IVCM examination, and/or the presence of either goblet cells or keratin-13 positive cells (14-16) by impression cytology were included in the current study from October 2010 to September 2018. The patients who had had prior limbal stem cell transplantation before the first visit to our institute were excluded. Those who could not cooperate and complete IVCM and AS-OCT examinations were excluded, too. Subjects who had no history of ocular surgery other than cataract surgery, contact lens wear, ocular trauma and chronic ocular cicatricial inflammatory diseases such as Stevens-Johnson syndrome were enrolled in the control group. Furthermore, all potential normal subjects underwent slit-lamp examination to exclude ocular surface abnormality such as pterygium before inclusion as normal controls. Demographic data that were collected included gender, date of birth, etiology of LSCD (principal diagnosis), other causes of decreased vision, best corrected visual acuity, and history of ocular surgery, ocular trauma, contact lens wear, and/or autoimmune diseases. The participants underwent a complete ophthalmic examination that included slit lamp biomicroscopy, AS-OCT, and IVCM. The measurements obtained using AS-OCT were included after December 2015 when the device became available.

### Image Acquisition

### Slit Lamp Photography

Slit lamp photographs of the central cornea and superior, inferior, nasal, and temporal limbus were taken under white light and cobalt blue light. The abnormal conjunctival/metaplastic epithelium was visualized with delayed fluorescein staining.

### AS-OCT Imaging

A Fourier-domain OCT system (RTVue-100; Optovue Inc, Fremont, Calif.) with a corneal module long adaptor lens (1.96-mm scan depth and 6-mm scan width) was used as previously described.(9, 17) The system's working wavelength was 830 nm, and the scan speed was 26,000 axial scans per second. Pachymetry scan mode and cross-line scan mode were used to obtain images of the central cornea. The subjects were asked to look upward, downward, to the left side, and to the right side by fixating at a peripheral target, and the limbal images of each quadrant (superior, inferior, nasal, and temporal) were obtained by using the cross-line scan mode. At least 3 scans using each mode were performed at both central cornea and each limbal quadrant.

### IVCM Imaging

IVCM of the central cornea and superior, inferior, nasal, and temporal limbus was performed by using a Heidelberg Retina Tomograph III Rostock Corneal Module confocal microscope (Heidelberg Engineering GmBH, Dossenheim, Germany) as previously described.(8) The subjects were asked to fixate at a light source with the fellow eye to minimize eye movement during image acquisition. At each location, 3 sequence scans and at least 3 volume Z-scans were obtained. A serial of volume Z-scans were taken to include the first layer of the epithelium (superficial layer) and the last layer of the epithelium (basal layer). Three best-focused images with minimal motion artifacts were selected from three different scans and were used for analysis.

### Image Analysis

### Clinical Subscore

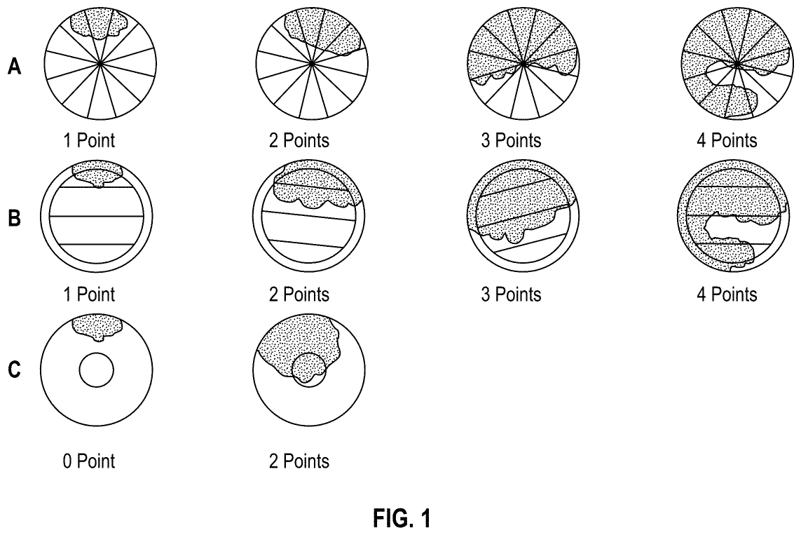

The distribution of abnormal conjunctival/metaplastic epithelium was evaluated on the basis of fluorescein staining and slit lamp findings, and a clinical score was obtained as previously reported.(18) In brief, the clinical score was composed of 3 parts: limbal involvement, corneal involvement, and visual axis involvement. Each part was assigned a score according to the schematic diagram shown in FIG. 1. Based on these 3 scores, the total clinical subscore (range, 1-10 points) was calculated, and the clinical stage of LSCD was determined: mild LSCD was indicated by a total subscore of 1-4 points; moderate LSCD, by a total score of 5-7 points, and severe LSCD, by a total score of 8-10 points. These clinical staging subscore correspond to the global consensus staging system.(3)

### Cell Morphology Analysis and Basal Cell Density

Cell morphology and BCD were analyzed in the confocal microscopic images of the central cornea and 4 limbal areas (superior, inferior, nasal, and temporal). At least three of the best, focused images of the basal epithelial layer at each location were selected from 3 different scans. The determination of basal epithelial cells was described in our earlier reports.(12, 13) The cell morphology in each image was evaluated and assigned a score based on the criteria shown in Supplementary FIG. 1 of Le et al., Ocul Surf 2022 Jan;23:123-130.

The measurement of BCD was performed by using the manufacturer's cell counting software as previously described.(8, 12) In brief, a frame was chosen in each image with the least compression artifacts, and the number of cells in this frame was highlighted. If the cells crossed the edges of the frame, only those crossing either upper or lower, and either left or right edges of the frame would be counted. Cell density was calculated automatically by the software. To ensure the reliability of the results, the frame had to be large enough to contain a minimum of 50 cells; the only exception was in cases in which the total number of cells in the entire image was <50. The measurements were performed by 2 masked independent observers, and the averages of their results were calculated.

### Corneal Epithelial Thickness (CET)

The images taken by AS-OCT were used to measure the thickness of corneal epithelium in the central 2-mm diameter zone. The customized software of the AS-OCT system automatically measured CET of normal eyes through the pachymetry scan mode. CET was manually measured on cornea cross-sectional scan images of eyes with LSCD as previously reported.(9) The mean value of the 3 measurements was then used to determine the CET. The measurements were performed by 2 masked independent observers, and the averages of their results were calculated.

### Limbal Epithelial Thickness (LET)

The measurement of LET included the mean LET and the maximum LET. The maximum LET was measured manually with the built-in measuring tool on AS-OCT images. The measurement of the mean LET was performed by using the method described previously.(9, 17) Measurements in each quadrant on at least 3 different scans were made, and the average values were calculated. All measurements were made by 2 masked, independent investigators.

### Analysis of the Subbasal Nerve Plexus

The subbasal nerve plexus shown on confocal microscopic images of the central cornea were analyzed. For each patient, at least 3 best-focused images were chosen from 3 different scans. A custom-designed, semiautomated nerve analysis software package CCMetrics (M. A. Dabbah, imaging science and biomedical engineering, University of Manchester, Manchester, United Kingdom) (19, 20) was used. Four parameters of the subbasal nerve plexus were provided: corneal nerve fiber length (CNFL: total length of main nerves and nerve branches per square millimeter), corneal nerve fiber density (CNFD: total number of main nerves per frame), corneal nerve branch density (CNBD: total number of branches per frame), and nerve tortuosity coefficient.(21) Two masked, independent observers made the measurements.

### Statistical Analysis

Summary statistics (mean, standard deviation, range, and frequency) were generated for the subjects' demographic and clinical information to characterize the study population. To compare variables between the control subjects and subjects with LSCD, the 2 sample t-test or Wilcoxon rank sum test was used for continuous variables, and the chi-square test or Fisher's exact test was used for categorical variables. Heatmaps were generated using ‘corrplof’ package in R software for data visualization to show the strength of Spearman correlations among biomarkers and LSCD disease severity. The size of the dot and the color variation in heatmap gives visual impression about how the strength of correlation is clustered or varies among variables. Then, among the variables with the strongest correlation, logistic regression and linear regression analyses were used to evaluate how well each biomarker can predict LSCD vs normal eyes and predict clinical score among eyes with LSCD. ROC curve analysis was performed for each biomarker to determine thresholds and corresponding specificity and sensitivity to separate eyes with LSCD and those without. All analyses were performed with R software (www.r-project.org).

### Results

A total of 126 subjects (172 eyes) with LSCD and 67 control subjects (99 eyes) without LSCD were included. The etiologies of LSCD included iatrogenic injury (84 eyes), contact lens wear (36 eyes), chronic cicatricial ocular surface inflammation (mucous membrane pemphigoid, 18 eyes; Stevens-Johnson syndrome, 6 eyes; other types of chronic ocular surface inflammation, 5 eyes), chemical injury (9 eyes), idiopathic cause (8 eyes), aniridia (5 eyes), and neurotrophic keratopathy (1 eye). The iatrogenic causes included multiple glaucoma surgeries with or without the use of anti-metabolites such as mitomycin C and 5-fluorouracil (51 eyes), multiple cryotherapies (12 eyes), radiation keratopathy (9 eyes), systematic chemotherapy (4 eyes), and multiple ocular surgeries (other than glaucoma surgery) combined with either cryotherapies or the use of mitomycin C (8 eyes). All subjects were evaluated by slit lamp microscopy and IVCM, and 69 eyes with LSCD and 52 control eyes without LSCD were evaluated by AS-OCT. The control group consisted of a population with a mean better BCVA than the LSCD group did (Table 1 of Le et al., Ocul Surf 2022 Jan;23:123-130).

### Correlation and Regression Analysis of All Parameters

A total of 29 in vivo parameters were included in the analysis. The measurements of all in vivo parameters are shown in Table 2 of Le et al., Ocul Surf. 2022 January;23:123-130. The analysis on the inter-observer bias between 2 examiners showed good inter-observer consistency and little variation (<5%) except in the analysis on subbasal nerve plexus (Supplementary Table 1 in Le et al., Ocul Surf 2022 Jan;23:123-130).

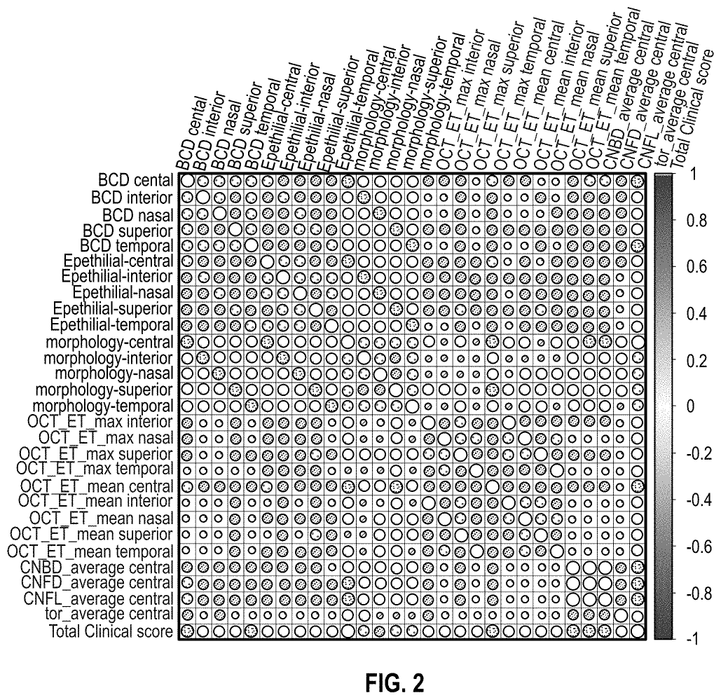

The heatmap (FIG. 2) is a color visualization of correlation analysis. FIG. 2A illustrates the correlation among variables in all study subjects, which showed that the clinical subscore had a strong positive correlation with cell morphology and a negative correlation with BCD and epithelial thickness in both the cornea and limbus, CNFL, and CNFD. Then the correlation analysis was performed in subjects with LSCD. As shown in FIG. 2B, the clinical subscore in LSCD eyes had a very strong negative correlation with only 3 parameters: BCD (ρ=−0.628, P<0.001), CET (ρ=−0.591, P=0.007), and CNFL (ρ=−0.555, P=0.010) in the central cornea. The clinical subscore also had a strong positive correlation with cell morphology at the central cornea (ρ=0.675, P<0.001; FIG. 2B). The visual axis involvement was positively correlated with the cell morphology score at the central cornea (ρ=0.593, P=0.002) and negatively correlated with the central cornea BCD (ρ=−0.603, P<0.001).

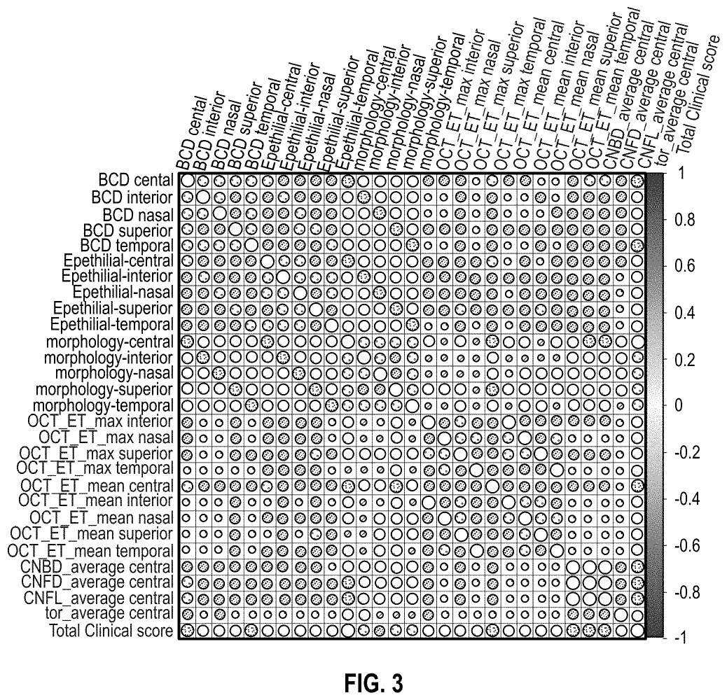

Next, a logistic regression analysis and linear regression analysis were performed to evaluate the effectiveness of all parameters to differentiate normal eyes from eyes with LSCD and to predict LSCD severity. The central cornea BCD, CET, and CNFL had the strongest correlation with clinical subscore (Coef=3.123, 3.379, and 2.223; P<0.001, <0.001, and =0.008, respectively), and were the best parameters for differentiating eyes with LSCD from those without (FIG. 3).

### Comprehensive Diagnostic and Staging System for LSCD

A comprehensive diagnostic and staging system based on the clinical subscore, central cornea BCD, CET, and CNFL was established. Receiver operating characteristic (ROC) curves were generated first, and the thresholds to distinguish normal eyes from eyes with LSCD were determined to be 7981 cells/mm² for BCD, 51 μm for CET, and 10,448 μm/mm² for CNFL; the specificity of the cut-off values for all parameters was 90% (OR=90%). The corresponding sensitivity was 92% for BCD, 93% for CET, and 86% for CNFL.

Next, tertiles, which are the two values separating the highest one third, the middle one third, and the lowest one third of data sample, were used to determine additional cut-off values of in vivo imaging parameters to classify LSCD severity. The method to assign the score for each parameter was presented in Table 3 of Le et al., Ocul Surf. 2022 January;23:123-130. The rank correlation analysis showed that the clinical score had similar positive correlations with the score for BCD at the central cornea (ρ=0.79), the score for CET (ρ=0.82), and the score for CNFL (ρ=0.71). Finally, taking the results of ROC curve, interobserver bias, regression analysis, and rank correlation analysis into consideration, we gave the score for central cornea BCD and the score for CET each a weight of 30% and the score for CNFL and the clinical score each a weight of 20%. The formula for the composite score was derived as follows: [(clinical score/3)*0.2+central cornea BCD score*0.3+CET score*0.3+CNFL score*0.2]*4. A comprehensive score ≥1 but <5 defined stage 1 LSCD, a comprehensive score ≥5 but <10 defined stage II LSCD, and a comprehensive score ≥10 defined stage III LSCD. We selected 2 cases to demonstrate the low reliability of clinical exam alone in the diagnosis and staging of LSCD. The proposed comprehensive scoring system provides a more accurate staging of LSCD.

### Case 1

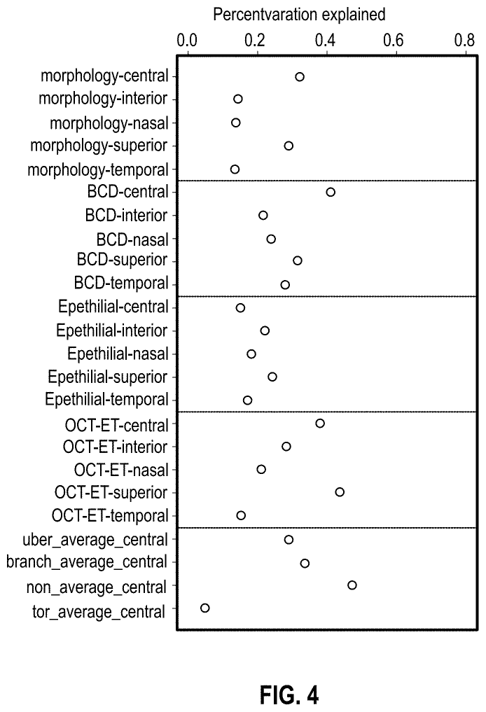

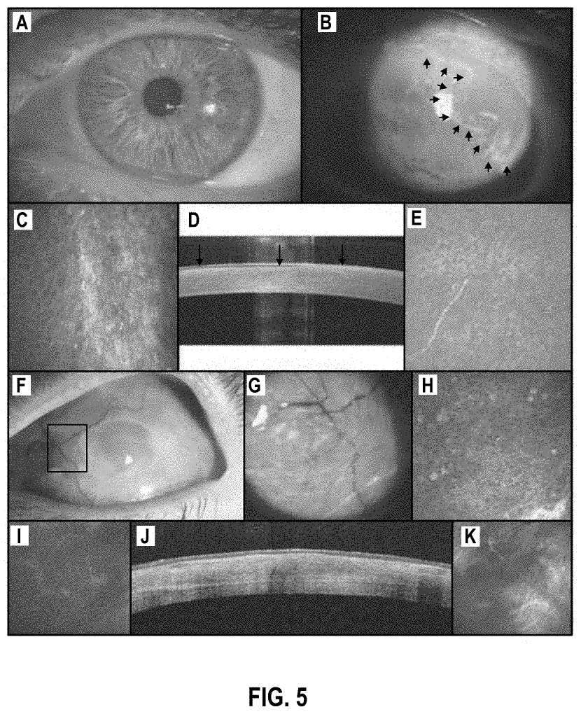

A 42-year-old white female with a history of contact lens wear for 20 years complained of ocular discomfort in her left eye. No gross abnormalities were found under white-light slit-lamp biomicroscopy. Fluorescein staining exam revealed late staining at the superior-temporal quadrant (FIG. 4A-E). The clinical grading for the limbal, corneal, and visual axis involvement were 2, 2, and 0, respectively. The total clinical subscore was 4, which was classified as stage I disease. However, IVCM showed abnormal cell morphology and the BCD, was 6398 cells/mm², the CET was 46 μm, and the CNFL was 8162 μm/mm² in the central cornea; these measures were then converted to a score of 2 from BCD, 2 from CET, and 1 from CNFL, respectively. The comprehensive score was 6.7; therefore, the disease was classified as stage II LSCD using the comprehensive staging method.

### Case 2

A 61-year-old white female with a history of decreased vision for 1 year presented with peripheral pannus, stromal opacity, and corneal neovascularization that had spared the central 2-mm cornea (FIG. 4F-K). Fluorescein uptake was visible only in the peripheral area of the nasal quadrant. The clinical scores for the limbal, corneal, and visual axis involvement were 3, 2, and 0, respectively, resulting in a total clinical subscore of 5. Based solely on this score, the severity was classified as stage II LSCD. However, IVCM showed a normal morphology of corneal epithelial cells on the central cornea, as well as normal limbal epithelial cells in the superior, inferior and temporal limbus. Abnormal epithelium and goblet cells were only detected at the nasal limbus and nasal peripheral cornea. In the central cornea, the BCD was 7901 cells/mm², the CET was 53 μm, and the CNFL was 10,377 μm/mm². Based on these values, the scores of each parameter were 1, 0, and 1, respectively. Therefore, the comprehensive score was 3.3, and severity of LSCD was reclassified as stage I.

### Discussion

Several studies have confirmed that clinical presentation alone may not be insufficient to make an accurate diagnosis and staging of LSCD particularly in complex cases.(5, 6, 22) Quantitative in vivo biomarkers have been used in the diagnosis of LSCD in recent years.(9, 18) The current study combined a clinical sub score and measurements of the in vivo biomarkers BCD, CET, and CNFL into a quantitative grading system. The establishment of such a comprehensive grading score could further reduce the bias caused by a single parameter and provide a more precise analysis and global assessment of the in vivo function of LSCD in normal eyes and those with LSCD. As demonstrated in the two cases, clinical presentation did not reflect the true LSC function. By taking into consideration all in vivo parameters, the in vivo LSC function i.e., the degree of LSCD could be more accurately quantified. This is particularly important in the decision making for surgical intervention and outcome measures.

Previous studies showed that the reduction of BCD, CET, and CNFL are signs of LSCD.(8, 9, 21, 23, 24) Their value in diagnosing LSCD has been confirmed by several studies.(25-27) Nevertheless, each of these in vivo parameters by itself might not be unique to LSCD, but the combination or composite of these parameters appear to be unique to LSCD. Although the central cornea BCD, CET, and CNFL may differentiate LSCD from other conditions, their weight in the current comprehensive grading system was different. The measurement of CNFL had a higher interobserver bias, and clinical presentation was likely to be underestimated or overestimated by inexperienced observers. Therefore, both of these parameters were assigned a weight of 20%. Measurements of the BCD and CET of the central cornea have greater consistency between observers and a relatively higher sensitivity, as the ROC curve showed. Hence, a weight of 30% was assigned to these 2 parameters. Of note the current study demonstrated that the clinical presentation of LSCD had a closer relationship with BCD, CET, CNFL, and cell morphology at the central cornea than with those of the limbal areas, indicating the importance of visual axis and central cornea in the evaluation of the severity of LSCD. In vivo biomarkers at the central cornea reflect the global function of LSCs.

The replacement of corneal epithelium by conjunctival epithelium is the hallmark of LSCD. According to the global consensus on definition, classification, diagnosis, and staging of LSCD,(3) phenotypic analysis of cells sampled from the corneal surface by impression cytology or IVCM images can be used confirm the diagnosis of LSCD. Although the presence of keratin 13⁺ cells detected by impression cytology is more sensitive than goblet cells to confirm the diagnosis of LSCD, neither of them correlates with the severity of LSCD and cannot be used for staging of the disease.(15) Therefore, the alteration of cell morphology from a corneal phenotype to a conjunctival phenotype is the key characteristic of LSCD. It has been reported that in mild to moderate LSCD, the nuclei of corneal basal epithelial cells become hyperreflective and prominent and these cells have a less distinctive border.(8) In severe LSCD, the entire corneal epithelial layer loses normal morphology and shows significant metaplasia.(22) The current study confirmed that the morphologic alterations of corneal basal epithelial cells are strongly and positively correlated with the clinical score. However, the evaluation of cell morphology is relatively more subjective than the assessments of the other parameters, and the results of the cell morphology evaluation depend on the experience of observers. Therefore, the score of cell morphology was not included in the final grading system in the current study. However, with the development of machine learning and artificial intelligence, the cell morphology seen on confocal microscopic images might be evaluated automatically by machine learning in the near future and thus overcome this limitation.

The proposed staging system is comprehensive, requiring multimodal in vivo imaging, a clinical laboratory for processing immunocytologic specimens, and experienced operators. IVCM might not be available in all tertiary eye centers. Therefore, this comprehensive scoring system would be best implemented in a tertiary eye center where both the technical and clinical expertise are available. However, the wider availability of high resolution AS-OCT around the world could be used as the initial screening tool by comprehensive ophthalmologists. Once epithelial thinning is confirmed to support the suspicion of LSCD, the patient could be referred to the eye care center where technical and clinical expertise are available. This approach is aligned with the recommendation of the global consensus of LSCD management.(7) The development of an online system for the upload, review, measurement, and sharing of images and the standardization of evaluating criteria for in vivo biomarkers might be a feasible way to facilitate the implementation of this staging system in the future.

In conclusion, significant microstructural changes are associated with LSCD and can be quantified by multimodal in vivo imaging such as IVCM and AS-OCT. Among all parameters measured by IVCM and AS-OCT, the central cornea BCD, CET and CNFL were found to have the highest positive correlation in differentiating normal corneas and eyes with LSCD. Compared with current diagnostic approaches, a comprehensive system that takes into consideration a clinical subscore and scores based on quantitative analyses of the central cornea BCD, CET, and CNFL could achieve a more accurate diagnosis and staging of LSCD before and after LSC transplantation.

### REFERENCES IN TEXT ABOVE

1. Tseng S C. Concept and application of limbal stem cells. Eye. 1989; 3 (Pt 2):141-57.
2. Cotsarelis G, Cheng S Z, Dong G, Sun T T, Lavker R M. Existence of slow-cycling limbal epithelial basal cells that can be preferentially stimulated to proliferate: implications on epithelial stem cells. Cell. 1989; 57(2):201-9.
3. Deng S X, Borderie V, Chan C C, Dana R, Figueiredo F C, Gomes J A P, et al. Global Consensus on Definition, Classification, Diagnosis, and Staging of Limbal Stem Cell Deficiency. Cornea. 2019; 38(3):364-75.
4. Le Q, Xu J, Deng S X. The diagnosis of limbal stem cell deficiency. The ocular surface. 2018; 16(1):58-69.
5. Chan E, Le Q, Codriansky A, Hong J, Xu J, Deng S X. Existence of Normal Limbal Epithelium in Eyes With Clinical Signs of Total Limbal Stem Cell Deficiency. Cornea. 2016; 35(11):1483-7.
6. Le Q, Samson C M, Deng S X. A Case of Corneal Neovascularization Misdiagnosed as Total Limbal Stem Cell Deficiency. Cornea. 2018; 37(8):1067-70.
7. Deng S X, Kruse F, Gomes J A P, Chan C C, Daya S, Dana R, et al. Global Consensus on the Management of Limbal Stem Cell Deficiency. Cornea. 2020; 39(10):1291-302.
8. Deng S X, Sejpal K D, Tang Q, Aldave A J, Lee O L, Yu F. Characterization of limbal stem cell deficiency by in vivo laser scanning confocal microscopy: a microstructural approach. Archives of ophthalmology. 2012; 130(4):440-5.
9. Liang Q, Le Q, Cordova D W, Tseng C H, Deng S X. Corneal Epithelial Thickness Measured Using AS-OCT as a Diagnostic Parameter for Limbal Stem Cell Deficiency. Am J Ophthalmol. 2020.
10. Nubile M, Lanzini M, Miri A, Pocobelli A, Calienno R, Curcio C, et al. In vivo confocal microscopy in diagnosis of limbal stem cell deficiency. American journal of ophthalmology. 2013; 155(2):220-32.
11. Binotti W W, Nose R M, Koseoglu N D, Dieckmann G M, Kenyon K, Hamrah P. The utility of anterior segment optical coherence tomography angiography for the assessment of limbal stem cell deficiency. The ocular surface. 2021; 19:94-103.
12. Chan E H, Chen L, Rao J Y, Yu F, Deng S X. Limbal Basal Cell Density Decreases in Limbal Stem Cell Deficiency. American journal of ophthalmology. 2015; 160(4):678-84 e4.
13. Chan E H, Chen L, Yu F, Deng S X. Epithelial Thinning in Limbal Stem Cell Deficiency. American journal of ophthalmology. 2015; 160(4):669-77 e4.
14. Poli M, Janin H, Justin V, Auxenfans C, Burillon C, Damour O. Keratin 13 immunostaining in corneal impression cytology for the diagnosis of limbal stem cell deficiency. Investigative ophthalmology & visual science. 2011; 52(13):9411-5.
15. Liang Q, Le Q, Wang L, Cordova D, Baclagon E, Garrido S G, et al. Cytokeratin 13 Is a New Biomarker for the Diagnosis of Limbal Stem Cell Deficiency. Cornea. 2021.
16. Ramirez-Miranda A, Nakatsu M N, Zarei-Ghanavati S, Nguyen C V, Deng S X. Keratin 13 is a more specific marker of conjunctival epithelium than keratin 19. Mol Vis. 2011; 17:1652-61.
17. Le Q, Cordova D, Xu J, Deng S X. In Vivo Evaluation of the Limbus Using Anterior Segment Optical Coherence Tomography. Transl Vis Sci Technol. 2018; 7(4):12.
18. Aravena C, Bozkurt K, Chuephanich P, Supiyaphun C, Yu F, Deng S X. Classification of Limbal Stem Cell Deficiency Using Clinical and Confocal Grading. Cornea. 2019; 38(1):1-7.
19. Efron N, Edwards K, Roper N, Pritchard N, Sampson G P, Shahidi A M, et al. Repeatability of measuring corneal subbasal nerve fiber length in individuals with type 2 diabetes. Eye & contact lens. 2010; 36(5):245-8.
20. Dabbah M A, Graham J, Petropoulos I N, Tavakoli M, Malik R A. Automatic analysis of diabetic peripheral neuropathy using multi-scale quantitative morphology of nerve fibres in corneal confocal microscopy imaging. Med Image Anal. 2011; 15(5):738-47.
21. Chuephanich P, Supiyaphun C, Aravena C, Bozkurt T K, Yu F, Deng S X. Characterization of the Corneal Subbasal Nerve Plexus in Limbal Stem Cell Deficiency. Cornea. 2017; 36(3):347-52.
22. Miri A, Alomar T, Nubile M, Al-Aqaba M, Lanzini M, Fares U, et al. In vivo confocal microscopic findings in patients with limbal stem cell deficiency. Br J Ophthalmol. 2012; 96(4):523-9.
23. Mehtani A, Agarwal M C, Sharma S, Chaudhary S. Diagnosis of limbal stem cell deficiency based on corneal epithelial thickness measured on anterior segment optical coherence tomography. Indian J Ophthalmol. 2017; 65(11):1120-6.
24. Tuck H, Park M, Carnell M, Machet J, Richardson A, Jukic M, et al. Neuronal-epithelial cell alignment: A determinant of health and disease status of the cornea. The ocular surface. 2021; 21:257-70.
25. Caro-Magdaleno M, Alfaro-Juarez A, Montero-Iruzubieta J, Fernandez-Palacin A, Munoz-Morales A, Castilla-Martino M A, et al. In vivo confocal microscopy indicates an inverse relationship between the sub-basal corneal plexus and the conjunctivalisation in patients with limbal stem cell deficiency. The British journal of ophthalmology. 2019; 103(3):327-31.
26. Wang L Y, Wei Z Y, Cao K, Su G Y, Liang Q F. [In vivo confocal microscopic characteristics of limbal stem cell deficiency]. Zhonghua Yan Ke Za Zhi. 2020; 56(6):447-55.
27. Bhattacharya P, Edwards K, Harkin D, Schmid K L. Central corneal basal cell density and nerve parameters in ocular surface disease and limbal stem cell deficiency: a review and meta-analysis. The British journal of ophthalmology. 2020; 104(12):1633-9.

### Example 2: Cell Morphology as an In Vivo Parameter for the

Diagnosis of Limbal Stem Cell Deficiency

Aspects of this disclosure are also found in: Bonnet et al., Cornea. 2022 Aug 1;41(8):995-1001.

The corneal epithelium plays a major role in maintaining corneal transparency, a prerequisite to visual function.¹ It is widely accepted that the maintenance and renewal of the corneal epithelium rely on stem cells located in the limbus (limbal stem cells; LSCs), which acts as a barrier to prevent conjunctivalization of the cornea.² Complex interactions between cells of the extracellular matrix, vessels, nerves, melanocytes, and signaling molecules control the homeostasis of LSCs.³ Their state of differentiation and proliferation is tightly regulated by their direct microenvironment, i.e., the limbal niche.⁴ In the human eye, the palisades of Vogt, the limbal crypts, and the limbal lacunae constitute the niche.⁵⁻⁸ Disturbance of the limbal niche by any negative factor such as genetic mutation, inflammation, or trauma can lead to the reduction or destruction of the LSC pool.² Hence, the maintenance of corneal epithelium homeostasis and barrier function are altered and invasion of conjunctival epithelial cells on the corneal surface occurs, thereby defining limbal stem cell deficiency (LSCD).

Classic clinical signs of LSCD include stippling or granular fluorescein staining of the metaplastic/conjunctival epithelium, which can be difficult to detect in early stages.² Other clinical signs can be nonspecific, including neurotrophic keratopathy, persistent epithelial defects, corneal neovascularization, haze, and chronic inflammation.² To objectively define and stage the disease, a recent global consensus has been established.⁹ Slit lamp examination and impression cytology have limitations as diagnostic methods; the diagnosis of LSCD may be confirmed by additional diagnostic tests such as in vivo laser scanning confocal microscopy (IVCM) or anterior segment optical coherence tomography.⁹

IVCM permits the visualization of central corneal parameters that correlate with disease severity and can be used to evaluate LSC function and stage LSCD. These IVCM parameters include the basal cell density (BCD), the epithelial thickness (ET), and the sub-basal corneal nerve fiber length density (CNFL) in the central cornea.¹⁰⁻¹⁴ Changes in cell morphology (CM) have also been observed in eyes with LSCD.¹⁵ In the mild stage of LSCD, basal epithelial cell borders become less distinct. During the moderate stage, the nuclei of these cells become more prominent, and in the severe stage the cells become enlarged and metaplastic.¹⁵ This study aims to investigate whether basal epithelial CM can be another in vivo parameter for use in assessing LSCD severity.

As discussed below, in order to investigate basal epithelial cell morphology (CM) in the central cornea and limbal areas of eyes with limbal stem cell deficiency (LSCD). We developed a CM scoring system based on basal epithelial cell phenotypes graded from 0 (normal) to 3 (severe morphologic alterations); this system was evaluated by 2 independent masked observers. The CM score was compared with the LSCD clinical score, the mean best corrected visual acuity (BCVA), and in vivo laser scanning confocal microscopy (IVCM) parameters used to stage the LSCD (i.e., basal epithelial cell density [BCD], basal epithelial thickness [ET], and sub-basal corneal nerve fiber length density [CNFL]). 168 eyes with LSCD and 63 normal eyes were included. Compared with the control group, the LSCD group had significantly higher mean (±SD) CM scores in the central cornea (1.8±0.7 vs 0.5±0.4, respectively; P=0.01) and limbal areas (1.6±0.2 vs 1.3±0.0, respectively; P<0.05). The mean CM score in the central cornea was positively correlated with the clinical score (P<0.01, r=0.66) and negatively correlated with the BCVA (P<0.01, r=0.42). The CM scores were positively correlated with all other IVCM parameters in the central cornea and limbal areas (all P<0.001). These studies show that basal epithelial CM is altered in the central cornea and limbus of eyes with LSCD and thus can be used to stage the clinical severity of the disease.

### Materials and Methods

This prospective, cross-sectional study was conducted at the Stein Eye Institute after the approval of the Institutional Review Board at the University of California, Los Angeles. Appropriate consent was obtained from study subjects per IRB protocol. The study was compliant with the HIPAA regulations and adhered to the Declaration of Helsinki.

Subjects with LSCD were consecutively recruited from the senior author's practice (S. X. D) between 2009 and 2017. The normal controls were recruited from the senior author's practice and the Comprehensive Division. The diagnosis of LSCD was based on clinical presentation, according to the criteria set by the International LSCD Working Group and confirmed by IVCM (HRT III, Heidelberg Engineering GmBH, Germany) and/or impression cytology.⁹ Impression cytology was performed for the 56 subjects with LSCD (33.3%) who were willing to undergo the test.¹⁶ All subjects with LSCD and 63 control subjects (63 eyes) underwent IVCM. Best corrected visual acuity (BCVA) using the Snellen chart was collected and converted to the logarithm of minimum angle of resolution (logMAR) for statistical analysis.

The stage of LSCD was classified as mild (2-4 points), moderate (5-7 points), or severe (8-10 points) based on the extent of corneal and limbal involvement defined by late stippling fluorescein staining, epithelial opacity with vortex pattern with or without epithelial defects, following a clinical scoring system previously described (See supplemental Figure in Bonnet et al., Cornea. 2022 Aug 1;41(8):995-1001).¹⁴ The phenotype of the epithelial cells was further confirmed by in vivo imaging. The mild, moderate, and severe stages are correlated with stages I, II, and III, respectively, established by the LSCD International Working Group.^{9, 17} The control group consisted of 10 eyes (15.9%) with, and 53 eyes (84.1%) without a history of contact lens wear. All control eyes were free of any ocular disease and any ocular surface abnormality that could have been detected by slit-lamp examination and had not undergone any ocular surgery other than cataract surgery.

### In Vivo Laser Scanning Confocal Microscopy

IVCM was performed on the central cornea and the 4 limbal areas (superior, inferior, nasal, and temporal).¹⁵ A minimum of 3 high-quality Z-scans were acquired in each area. Measurements were performed in the 5 areas of the basal epithelial layer, which was just above the sub-basal nerve plexus location. In vivo parameters of LSC function (BCD, ET, and CNFL) that were previously reported to correlate with the severity of the disease were collected for each area.¹⁰⁻¹² ET was defined as the scan depth difference between the most superficial layer of the epithelium and the basal layer.¹¹ BCD was measured as recommended by the manufacturer.¹⁵ CNFL was measured as the fiber length density φm/mm²), which was evaluated by ACCMetrics as previously described (semiautomated software. University of Manchester. UK).¹⁸

CM findings of the basal epithelial cells previously described in eyes with LSCD of differing severity were used to develop a staging system consisting of 4 grades.¹⁵ Morphologic criteria were the epithelial cell type (corneal or conjunctival), the intercellular cell border visibility, the cell body size and shape, the cytoplasm reflectivity, and the nucleus size and reflectivity (see Table 1 below and FIG. 1 in Bonnet et al., Cornea. 2022 Aug 1;41(8):995-1001).

### Statistical Analysis

The average value of 3 measurements by IVCM in each area was obtained. These measurements in addition to the LSCD clinical grading were performed by 2 independent masked observers. The intraclass correlation coefficient between the 2 observers was 0.89, which confirmed their high level of agreement. Correlations between the CM score, the clinical score, the BCVA, and the IVCM parameters were characterized by box plots and Spearman correlation coefficients with all subjects. To compare the correlation coefficients, a bootstrap method was used. Statistical analysis was performed by a biostatistician (C. H. T) using R software (www.r-project.org). Any P value<0.05 was considered statistically significant.

### Results

### Subject Demographics

Demographics of the 231 eyes included in the study (LSCD, 168 eyes; control, 63 eyes) are presented in Table 2 of Bonnet et al., Cornea. 2022 Aug 1;41(8):995-1001. The LSCD and control groups were comparable in terms of mean age and sex (all P>0.05). The mean BCVA was significantly lower in the LSCD group (P<0.01). The most frequent etiologies of LSCD were multiple ocular surgeries (85 eyes, 50.6%) and contact lens wear (37 eyes; 22.0%). LSCD stages, based on clinical scores, were mild in 63 eyes (37.5%), moderate in 55 eyes (32.7%), and severe in 50 eyes (29.8%).

### Cell Morphology

According to the CM scoring presented in FIG. 1 in Bonnet et al., Cornea. 2022 Aug 1;41(8):995-1001, CM in the central cornea had a score of 0 (normal) in 5 eyes (3.0%), of 1 (mild) in 76 eyes (45.2%), 2 (moderate) in 42 eyes (25.0%), and 3 (severe) in 45 eyes (26.8%). In the control group, the central cornea CM score was 0 (normal) in 41 eyes (65.1%) and 1 (mild) in 22 eyes (34.9%; Table 2 in Bonnet et al., Cornea. 2022 Aug 1;41(8):995-1001.). There was no difference in the central cornea CM score between control eyes with and without history of contact lens wear (P=0.16). The mean CM scores were significantly higher in the LSCD group than the control group in the central cornea (1.8±0.7 in the LSCD group vs 0.5±0.4 in the control group; P=0.01) and limbal areas (1.6±0.2 in the LSCD group vs 1.3±0.0 in the control group; P<0.05). The sensitivity and specificity of the scoring system using threshold values are presented in Table 3 of Bonnet et al., Cornea. 2022 Aug 1;41(8):995-1001.

Significant correlations were found between the CM score, clinical score, BCVA, and IVCM parameters (BCD, ET, and CNFL). A positive correlation was observed between the CM score and the clinical severity score in the central cornea (see FIG. 2A in Bonnet et al., Cornea. 2022 Aug 1;41(8):995-1001; P<0.01, r=0.79) and limbal areas (see FIG. 3A in Bonnet et al., Cornea. 2022 Aug 1;41(8):995-1001; P<0.01, r=0.72). The CM scores of both the central cornea (see FIG. 2B in Bonnet et al., Cornea. 2022 Aug 1;41(8):995-1001; P<0.01, r=0.61) and limbal areas (see FIG. 3B in Bonnet et al., Cornea. 2022 Aug 1;41(8):995-1001; P<0.01, r=0.64) were correlated positively with the BCVA. When the CM score of the central cornea was compared with the IVCM parameters, we also found strong negative correlations with the central cornea BCD (see FIG. 2C in Bonnet et al., Cornea. 2022 Aug 1;41(8):995-1001; P<0.01, r=−0.80), the central ET (see FIG. 2D in Bonnet et al., Cornea. 2022 Aug 1;41(8):995-1001; P<0.01, r=−0.61), and the central CNFL (see FIG. 2E in Bonnet et al., Cornea. 2022 Aug 1;41(8):995-1001; P<0.01, r=−0.71). The CM scores of all limbal areas were negatively correlated with BCD of all limbal areas (see FIG. 3C in in Bonnet et al., Cornea. 2022 Aug 1;41(8):995-1001; P<0.01, r=−0.80) and ET of all limbal areas (see FIG. 3D in Bonnet et al., Cornea. 2022 Aug 1;41(8):995-1001; P<0.01, r=−0.73). Comparison of the central cornea correlations coefficients with the clinical scores revealed that BCD, CNFL, and CM had higher correlations than ET (See supplemental Table 1 in Bonnet et al., Cornea. 2022 Aug 1;41(8):995-1001; P<0.05). No significant differences were found between the BCD, CNFL, and CM correlation coefficients. In the limbus, comparison of the correlation coefficients with the clinical score revealed that BCD and ET had higher correlations than CM (See supplemental Table 2 in Bonnet et al., Cornea. 2022 Aug 1;41(8):995-1001; P<0.05). No significant differences were found between BCD and CM correlation coefficients. Clinical examples of the CM scores in eyes with different stages of LSCD severity are presented in FIG. 4 of Bonnet et al., Cornea. 2022 Aug 1;41(8):995-1001.

### Discussion

A diagnosis of LSCD may be confirmed and the severity staged by using several biomarkers including BCD, ET, and CNFL.¹⁰⁻¹³ The current study shows that CM changes in the central cornea and the limbus of eyes with LSCD are positively correlated with other in vivo parameters, specifically BCD, ET, and CNFL.^{10-12, 14} Thus, CM is an additional biomarker that can be used to confirm the diagnosis and classify the severity of LSCD. Changes in epithelial CM observed using IVCM included the number of cell layers, cell size, and degree of reflectivity of the nucleus and the cell-cell junction.

CM changes and decreased cellular density are observed in other ocular surface diseases, such as dry eye diseases, keratoconus, vernal keratoconjunctivitis, and abnormalities after refractive surgeries.¹⁹⁻²³ However, in these diseases, the CM changes affect mostly the superficial corneal epithelial cells, nerves and anterior and/or posterior stromal keratocytes, whereas the basal epithelial cells remain largely unaffected. The CM changes of the basal epithelial cells described in this study are observed in LSCD.

Different morphologic features have been previously described to assess the epithelial phenotypes such as corneal, conjunctival, or mixed on the cornea surface.^{15, 24-27}. The phenotype of the epithelial cells identified by IVCM has also been confirmed by impression cytology.^{24, 26, 27} Lagali et al. reported that progression of LSCD in aniridia correlated with gradual loss of palisades structures, corneal epithelial cell phenotype, and corneal nerve.²⁵ Miri et al. reported that cell size and density was decreased in eyes with LSCD.²⁶ Shortt et al. developed a CM scoring system using 3 criteria: absence of epithelial cells visible; non-stratified epithelium 1 or 2 layers thick, with hyperreflective nuclei but loss of intercellular junctions; and stratified epithelium with clear intercellular boundaries indicating normal epithelial function. The study was able to correlate the morphologic presentation with phenotypic marker of conjunctival (cytokeratin 19) or corneal (cytokeratin 3) markers up to 3 years after transplantation of cultivated allogeneic limbal epithelial cells.²⁴ However, these studies remained descriptive, without providing correlations between the CM changes, the clinical stage and other in vivo parameters (BCD, ET, and CNFL). By providing such correlations, the current study further confirms CM as a biomarker of LSCD severity.

These in vivo biomarkers can also be used to evaluate the success of LSC transplantation. For example, Borderie et al. used the BCD to evaluate the success (i.e. the absence of recurrence of clinical signs of LSCD) of different type of LSC transplantation in eyes with stage III LSCD.²⁸ Three years after transplantation, a higher BCD (6558 cells/mm² in average) was observed in success cases. In an ongoing phase I clinical trial (NCT03957954) that investigates the safety and feasibility of cultivated autologous LSC for LSCD, all 4 biomarkers, clinical scores, ET, BCD, CNFL, and CM are being used to assess the LSC function.

Significant BCD and ET reduction are early signs of LSC dysfunction and are correlated with the severity of LSCD.^{10, 11, 15} The correlation found between CM scoring and mild LSCD suggests that CM changes are also early findings of corneal epithelial dysfunction, which could be more objective than the subtle early clinical signs.¹⁴ An inverse relationship between the reduction in BCD and the basal cell size diameter has been previously described.¹⁰ Using our CM scoring system, we found that the central CM score had the strongest correlation with the central BCD than with ET or CNFL. The most relevant IVCM parameter to characterize LSC function remains to be determined. Each parameter has advantages and limitations. ET is a relatively objective measure. ET measured by anterior segment optical coherence tomography is a widely accessible, non-contact test that correlates with the severity of LSCD, making it a good screening tool for general ophthalmologists.′ Compared with ET or CNFL, preliminary results show that BCD is better correlated with disease severity in both the central cornea and limbus.^{10, 12} Current BCD analysis remains manual, time-consuming, and requires experienced observers, thus limiting its use to eye care centers with expertise in this type of analysis.

CNFL is another major criterion correlated with LSCD severity.^{12, 29, 30} Close interactions between basal corneal epithelial cells and nerves are necessary to support the physiologic secretion of nerve growth factors.^{31, 32} The loss of these interactions affect the maintenance of healthy nerves, corneal epithelial cells, and LSCs.^{4, 12, 31, 32} Similar to BCD, CNFL analysis requires more sophisticated software and experienced observers.¹² Other limitations include compression artifacts that can occur during the scan acquisition and the presence of hyperreflective corneal scarring often seen in the severe stage of LSCD. CM is a more subjective analysis than the analyses of other IVCM parameters as evaluation of CM requires the knowledge of recognizing the cell morphologic phenotypes (corneal, metaplastic, or conjunctival). Machine deep-learning is a promising approach that enables automated and more objective quantification of these in vivo parameters.³³⁻³⁵ Further studies are necessary to evaluate the weight of each biomarker to determine which one has a more accurate diagnostic value. It is likely that evaluation of a combination of all the IVCM parameters will be needed to obtain a comprehensive evaluation of LSC function.⁹

In summary, the CM score correlates with the severity of the LSCD and is an IVCM parameter that can aid in the diagnosis and staging of the disease.

TABLE 1
CM Score Based on IVCM Findings

| IVCM Criteria | Normal-Grade 0 | Mild-Grade 1 | Moderate-Grade 2 | Severe-Grade 3* |
|---|---|---|---|---|
| Intercellular cell border | Distinct | Blurry | Not visible | Not visible |
| Cell body size shape | Small-regular | Small-regular | Enlarged-irregular | Large-loose |
| Cytoplasm reflectivity | Hyporeflective | Hypo/hyperreflective | Hypo/hyperreflective | Hyperreflective |
| Nucleus size-reflectivity | Not visible | Small-hyperreflective | Large-hyperreflective | Not visible |
| | | (low N/C ratio) | (high N/C ratio) | |

*When cells were not visible, score was considered severe, grade 3.
N/C ratio, nucleus/cytoplasm ratio.

### Example 2 References

1. Thoft, R A, Friend, J. The X, Y, Z hypothesis of corneal epithelial maintenance. Investigative ophthalmology & visual science 1983; 24:1442-3.
2. Le, Q, Xu, J, Deng, S X. The diagnosis of limbal stem cell deficiency. Ocul Surf 2018; 16:58-69.
3. Bonnet, C, Gonzalez, S, Roberts, J S, et al. Human limbal epithelial stem cell regulation, bioengineering and function. Prog Retin Eye Res 2021:100956.
4. Seyed-Safi, A, Daniels, J. The limbus: Structure and function. Experimental eye research 2020; 197:108074.
5. Goldberg, M F, Bron, A J. Limbal palisades of Vogt. Trans Am Ophthalmol Soc 1982; 80:155-71.
6. Shortt, A J, Secker, G A, Munro, P M, et al. Characterization of the limbal epithelial stem cell niche: novel imaging techniques permit in vivo observation and targeted biopsy of limbal epithelial stem cells. Stem cells 2007; 25:1402-9.
7. Dua, H S, Shanmuganathan, V A, Powell-Richards, A O, et al. Limbal epithelial crypts: a novel anatomical structure and a putative limbal stem cell niche. Br J Ophthalmol 2005; 89:529-32.
8. Zarei-Ghanavati, S, Ramirez-Miranda, A, Deng, S X. Limbal lacuna: a novel limbal structure detected by in vivo laser scanning confocal microscopy. Ophthalmic Surg Lasers Imaging 2011; 42 Online: e129-31.
9. Deng, S X, Borderie, V, Chan, C C, et al. Global Consensus on Definition, Classification, Diagnosis, and Staging of Limbal Stem Cell Deficiency. Cornea 2019; 38:364-375.
10. Chan, E H, Chen, L, Rao, J Y, et al. Limbal Basal Cell Density Decreases in Limbal Stem Cell Deficiency. Am J Ophthalmol 2015; 160:678-84.
11. Chan, E H, Chen, L, Yu, F, et al. Epithelial Thinning in Limbal Stem Cell Deficiency. Am J Ophthalmol 2015; 160:669-677.
12. Chuephanich, P, Supiyaphun, C, Aravena, C, et al. Characterization of the Corneal Subbasal Nerve Plexus in Limbal Stem Cell Deficiency. Cornea 2017; 36:347-352.
13. Liang, Q, Le, Q, Cordova, D W, et al. Corneal Epithelial Thickness Measured Using Anterior Segment Optical Coherence Tomography as a Diagnostic Parameter for Limbal Stem Cell Deficiency. Am J Ophthalmol 2020; 216:132-139.
14. Aravena, C, Bozkurt, K, Chuephanich, P, et al. Classification of Limbal Stem Cell Deficiency Using Clinical and Confocal Grading. Cornea 2019; 38:1-7.
15. Deng, S X, Sejpal, K D, Tang, Q, et al. Characterization of limbal stem cell deficiency by in vivo laser scanning confocal microscopy: a microstructural approach. Arch Ophthalmol 2012; 130:440-5.
16. Ramirez-Miranda, A, Nakatsu, M N, Zarei-Ghanavati, S, et al. Keratin 13 is a more specific marker of conjunctival epithelium than keratin 19. Mol Vis 2011; 17:1652-61.
17. Sun, Y, Yung, M, Huang, L, et al. Limbal Stem Cell Deficiency After Glaucoma Surgery. Cornea 2020; 39:566-572.
18. Tavakoli, M, Ferdousi, M, Petropoulos, I N, et al. Normative values for corneal nerve morphology assessed using corneal confocal microscopy: a multinational normative data set. Diabetes Care 2015; 38:838-43.
19. Villani, E, Galimberti, D, Viola, F, et al. The cornea in Sjogren's syndrome: an in vivo confocal study. Investigative ophthalmology & visual science 2007; 48:2017-22.
20. Leonardi, A, Lazzarini, D, Bortolotti, M, et al. Corneal confocal microscopy in patients with vernal keratoconjunctivitis. Ophthalmology 2012; 119:509-15.
21. Linna, T, Tervo, T. Real-time confocal microscopic observations on human corneal nerves and wound healing after excimer laser photorefractive keratectomy. Curr Eye Res 1997; 16:640-9.
22. Matsumoto, Y, Ibrahim, O M A, Kojima, T, et al. Corneal In Vivo Laser-Scanning Confocal Microscopy Findings in Dry Eye Patients with Sjögren's Syndrome. Diagnostics (Basel) 2020;10:497-500.
23. Benitez del Castillo, J M, Wasfy, M A, Fernandez, C, et al. An in vivo confocal masked study on corneal epithelium and subbasal nerves in patients with dry eye. Investigative ophthalmology & visual science 2004; 45:3030-5.
24. Shortt, A J, Bunce, C, Levis, H J, et al. Three-year outcomes of cultured limbal epithelial allografts in aniridia and Stevens-Johnson syndrome evaluated using the Clinical Outcome Assessment in Surgical Trials assessment tool. Stem Cells Transl Med 2014; 3:265-75.
25. Lagali, N, Eden, U, Utheim, T P, et al. In vivo morphology of the limbal palisades of vogt correlates with progressive stem cell deficiency in aniridia-related keratopathy. Investigative ophthalmology & visual science 2013; 54:5333-42.
26. Miri, A, Alomar, T, Nubile, M, et al. In vivo confocal microscopic findings in patients with limbal stem cell deficiency. Br J Ophthalmol 2012; 96:523-9.
27. Nubile, M, Lanzini, M, Miri, A, et al. In vivo confocal microscopy in diagnosis of limbal stem cell deficiency. Am J Ophthalmol 2013; 155:220-32.
28. Borderie, V M, Ghoubay, D, Georgeon, C, et al. Long-Term Results of Cultured Limbal Stem Cell Versus Limbal Tissue Transplantation in Stage III Limbal Deficiency. Stem Cells Transl Med 2019; 8:1230-1241.
29. Kadar, T, Dachir, S, Cohen, M, et al. Prolonged impairment of corneal innervation after exposure to sulfur mustard and its relation to the development of delayed limbal stem cell deficiency. Cornea 2013;32:e44-50.
30. Caro-Magdaleno, M, Alfaro-Juarez, A, Montero-Iruzubieta, J, et al. In vivo confocal microscopy indicates an inverse relationship between the sub-basal corneal plexus and the conjunctivalisation in patients with limbal stem cell deficiency. Br J Ophthalmol 2019; 103:327-331.
31. You, L, Kruse, F E, Volcker, H E. Neurotrophic factors in the human cornea. Investigative ophthalmology & visual science 2000; 41:692-702.
32. Tuck, H, Park, M, Camell, M, et al. Neuronal-epithelial cell alignment: A determinant of health and disease status of the cornea. Ocul Surf 2021.
33. Allgeier, S, Zhivov, A, Eberle, F, et al. Image reconstruction of the subbasal nerve plexus with in vivo confocal microscopy. Invest Ophthalmol Vis Sci 2011; 52:5022-8.
34. Prakasam, R K, Winter, K, Schwiede, M, et al. Characteristic quantities of corneal epithelial structures in confocal laser scanning microscopic volume data sets. Cornea 2013; 32:636-43.
35. Bohn, S, Sperlich, K, Allgeier, S, et al. Cellular in vivo 3D imaging of the cornea by confocal laser scanning microscopy. Biomed Opt Express 2018; 9:2511-2525.

All publications mentioned herein (e.g., those cited above) are incorporated by reference to disclose and describe aspects, methods and/or materials in connection with the cited publications. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

1. A method of observing the presence, absence or stage of limbal stem cell deficiency (LSCD) in a patient comprising:
(a) in the patient observing at least one of:
central corneal basal cell density;
limbal basal cell density;
central corneal epithelial layer thickness;
mean limbal epithelial layer thickness;
maximum limbal epithelial layer thickness;
total corneal nerve fiber length;
corneal nerve fiber density;
corneal nerve branch density;
basal epithelial cell morphology; and
nerve tortuosity coefficient; and
(b) correlating the observation of (a) with the presence or absence or stage of LSCD in the patient; wherein:
an increase in a LSCD clinical score correlates with a decrease in central corneal basal cell density, limbal basal cell density, corneal epithelial layer thickness, mean limbal epithelial layer thickness, maximum limbal epithelial layer thickness, total corneal nerve fiber length, corneal nerve fiber density, corneal nerve branch density, and nerve tortuosity coefficient;
so that the presence or absence or stage of LSCD in a patient is observed.

2. The method of claim 1, wherein the method comprises correlating observations of basal cell density, corneal epithelial thickness and total corneal nerve fiber length with the presence or absence or stage of LSCD in the patient.

3. The method of claim 1, wherein the method comprises observing at least: limbal basal cell density; epithelial layer thickness; basal epithelial cell morphology; or corneal nerve branch density.

4. The method of claim 1, wherein the method comprises observing at least 2 or 3 of: limbal basal cell density; epithelial layer thickness; basal epithelial cell morphology; or corneal nerve branch density.

5. The method of claim 1, wherein the method comprises observing a clinical score, a central cornea basal cell density score, a corneal epithelial thickness score and a total corneal nerve fiber length score.

6. The method of claim 5, wherein the method derives a comprehensive clinical score using a formula as follows:
[(clinical score/3)*0.2+central cornea basal cell density score*0.3+corneal epithelial thickness score*0.3+total corneal nerve fiber length score*0.2]*4; wherein:
a comprehensive score ≥1 but <5 defines stage 1 LSCD;
a comprehensive score ≥5 but <10 defines stage II LSCD; and
a comprehensive score ≥10 defines stage III LSCD.

7. The method of claim 1, wherein the method comprises correlating the observation of (a) with the stage of LSCD in the patient.

8. The method of claim 1, wherein the method comprises observing at least one of impression cytology images, anterior segment optical coherence tomography images or in vivo laser scanning confocal microscopy images obtained from the patient.

9. The method of claim 1, further comprising administering a therapeutic agent to a patient observed to exhibit the presence of limbal stem cell deficiency.

10. The method of claim 9, wherein the therapeutic agent comprises vitamin A, a methylprednisolone, a loteprednol etabonate, a prednisolone acetate, and/or a cyclosporine.

11. A limbal stem cell deficiency (LSCD) diagnostic system comprising:
a processor;
a computer-readable program having instructions which cause the processor to:
(a) assess data obtained from a patient comprising:
central corneal basal cell density;
limbal basal cell density;
central corneal epithelial layer thickness;
mean limbal epithelial layer thickness;
maximum limbal epithelial layer thickness;
total corneal nerve fiber length;
corneal nerve fiber density;
corneal nerve branch density;
basal epithelial cell morphology; and
nerve tortuosity coefficient; and
(b) correlate the observation of (a) with the presence or absence or stage of LSCD in the patient; wherein:
an increase in a LSCD clinical score correlates with a decrease in central corneal basal cell density, limbal basal cell density, corneal epithelial layer thickness, mean limbal epithelial layer thickness, maximum limbal epithelial layer thickness, total corneal nerve fiber length, corneal nerve fiber density, corneal nerve branch density, and nerve tortuosity coefficient.

12. The system of claim 11, wherein the processor uses an algorithm to calculate a LSCD clinical score.

13. The system of claim 12, wherein the algorithm derives a comprehensive clinical score using a formula as follows:
[(clinical score/3)*0.2+central cornea basal cell density score*0.3+corneal epithelial thickness score*0.3+total corneal nerve fiber length score*0.2]*4; wherein:
a comprehensive score ≥1 but <5 defines stage 1 LSCD;
a comprehensive score ≥5 but <10 defines stage II LSCD; and
a comprehensive score ≥10 defines stage III LSCD.

14. The system of claim 12, wherein the processor uses an algorithm to identify one or more treatment options for a patient having the calculated LSCD score.

15. The system of claim 15, the one or more treatment options comprise administration of a composition comprising vitamin A, a methylprednisolone, a loteprednol etabonate, a prednisolone acetate, and/or a cyclosporine.
